(19) United States
(12) Patent Application Publication
(10) Pub. No.: US 2014/0301900 A1
(43) Pub. Date: Oct. 9, 2014

(54) APPARATUS FOR DISINFECTING AND SANITIZING BRUSHES

(71) Applicant: Jose Piccin Germano, Sao Carlos (BR)

(72) Inventor: Jose Piccin Germano, Sao Carlos (BR)

(73) Assignee: BIO-ART EQUIPAMENTOS ODONTOLOGICOS LTDA, Sao Cantos, SP (BR)

(21) Appl. No.: 14/000,016

(22) PCT Filed: Feb. 13, 2012
(86) PCT No.: PCT/BR2012/000041

§ 371 Date: Aug. 27, 2013

(30) Foreign Application Priority Data
Feb. 18, 2011 (BR) PI1100260-3

(51) Int. Cl. A61L 2/20; A61L 2/26

(52) U.S. Cl. CPC A61L 2/20; A61L 2/26
USPC 422/114; 422/300; 422/119

(57) ABSTRACT

An apparatus (100, 200) is described for disinfecting and sanitizing up to three brushes (16) with steam, where the brushes (16) are placed in a horizontal position in the apparatus, such that the handles (16a) of the brushes (16) protrude from the apparatus and are not exposed to the steam generated for disinfecting and sanitizing the brushes. The apparatus (100, 200) comprises a case (1) covered by a tilting lid (8) and supported by a base (2), a water tank (3) and a chamber (4) located inside the case (1), the chamber (4) having a system for evaporating the water contained inside the tank (3), a support (5) for the brushes (16), a resistance heating system (6) for heating the water, a protection screen (7), a thermostat (26) and an electronic or mechanical control and operation system.

### FIELD OF THE INVENTION

The present application relates to the field of sanitizing and disinfecting apparatuses of brushes, combs, instruments and accessories in general, more specifically to an apparatus able to provide the microorganisms removal from various brushes, using the steam as action factor.

### BACKGROUND OF THE INVENTION

There are known some patent documents which disclose products of sanitizing and disinfecting brushes, combs, instruments and accessories in order to eliminate bacteria and microorganisms, preventing the transmission of capillary diseases.

The International Publication WO2005046389A1, (corresponding to IT2003T0000908) discloses an apparatus for cleaning and sanitizing brushes, where the brushes are arranged in a housing which remains in the upright position and so there is in a first step the removal of the hair present on the brush and following the contact of the steam with the brush the disinfection is performed. But the handle of the brush and comb are also in contact with the steam, so they are warmed, causing accidents. The system still has rotary movements for brushes, increasing the product price.

The Brazilian application PI0605466-8, from the same holder or applicant of the present application is a sanitizer apparatus for disinfecting and sanitizing hair brushes, brushes for veterinary use or similar instruments. However, this document presents a sanitizer which enables disinfecting a brush a time and, moreover, the handle of brushes, combs, among others, is arranged vertically to the sanitizing chamber, and it is inevitable the contact of the steam with the handle of these instruments and the consequent damage to them.

In this sense, the concept of the invention object of the present application represents a technological progress in relation to the product disclosed by the document object of the Brazilian application PI0605466-8 since it allows to perform the brushes disinfecting and sanitizing in greater amount and with brushes handles and other products to be disinfected and sanitized facing the outside of the apparatus, avoiding any heating due to the steam in plastic or wooden handles of the brushes or combs.

The document WO2010/035290, in turn, discloses an apparatus to clean only one brush at a time, being composed of an eliminator of hair in the brush and steam to disinfect the same.

It would therefore be interesting if the art had a technical apparatus able to disinfect and sanitize more than one brush, comb and/or instruments and accessories at a time, in up to six minutes, so still preserving the handles of these brushes, combs and/or instruments and accessories.

### SUMMARY OF THE INVENTION

In a wide way, the apparatus of the invention for disinfecting and sanitizing brushes, combs, instruments and accessories comprises one case upon a base having supports, tank for water to be vaporized, chamber for steaming, support for brushes, electric resistance heating system, protection screen, tilting lid, thermostat, electronic system of control and operation provided with electronic circuit board, transformer, fuse holder, power cable, on/off switch, blue and red LEDs and sensor.

Alternatively, the apparatus is presented in a mechanical version without using LEDs and other electronic devices.

When turning on the apparatus, the water contained in the chamber is heated, generating steam that involves the brushes. After six minutes the steam in contact with the brushes cleans thereof.

Thus, the present invention provides an apparatus for disinfecting and sanitizing brushes, the apparatus able to disinfect and sanitizes three brushes simultaneously.

The present invention also provides an apparatus for disinfecting and sanitizing brushes such that the brushes handles are not affected by the steam used for disinfecting and sanitizing.

The present invention also provides an apparatus for disinfecting and sanitizing brushes where the disinfecting and sanitizing is performed only by steam.

The present invention additionally provides an apparatus for disinfecting and sanitizing brushes, combs, instruments and accessories, the apparatus able to perform the disinfecting and sanitizing in about six minutes.

The present invention additionally provides an apparatus for disinfecting and sanitizing brushes in an electronic version.

The present invention also provides an apparatus for disinfecting and sanitizing brushes in a mechanic version.

### BRIEF DESCRIPTION OF THE FIGURES

FIG. 1 illustrates an expanded right perspective view of the apparatus of the invention in the electronic version showing its component parts.

FIG. 2 illustrates the apparatus of the invention in the electronic version in perspective view highlighting the right side thereof.

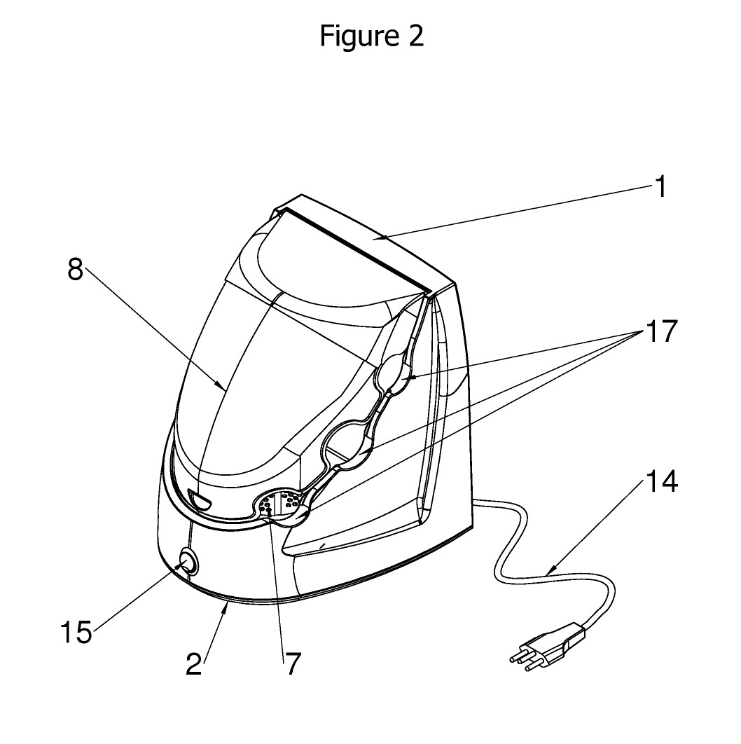

FIG. 3 illustrates the apparatus of the invention in the electronic version in perspective view, highlighting the left side thereof.

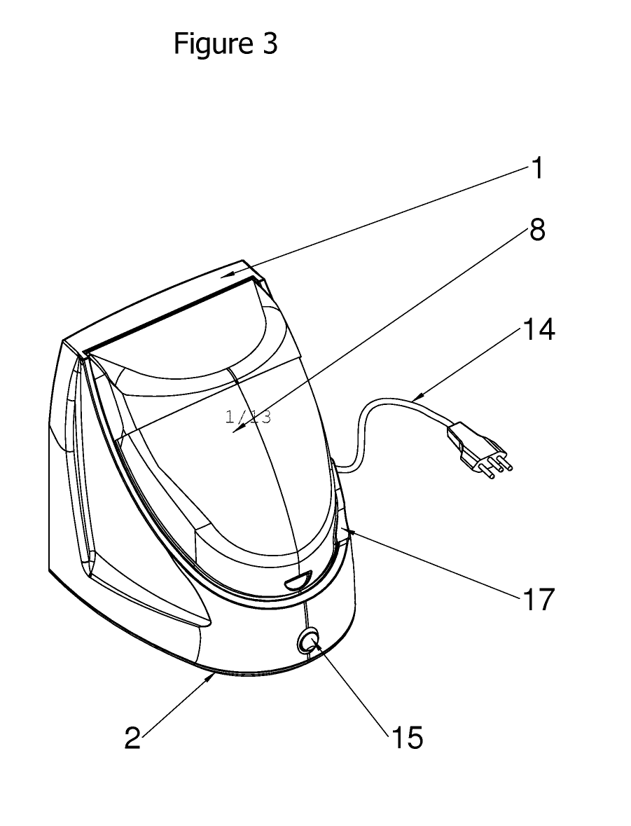

FIG. 4 illustrates the apparatus of the invention in the electronic version in front view.

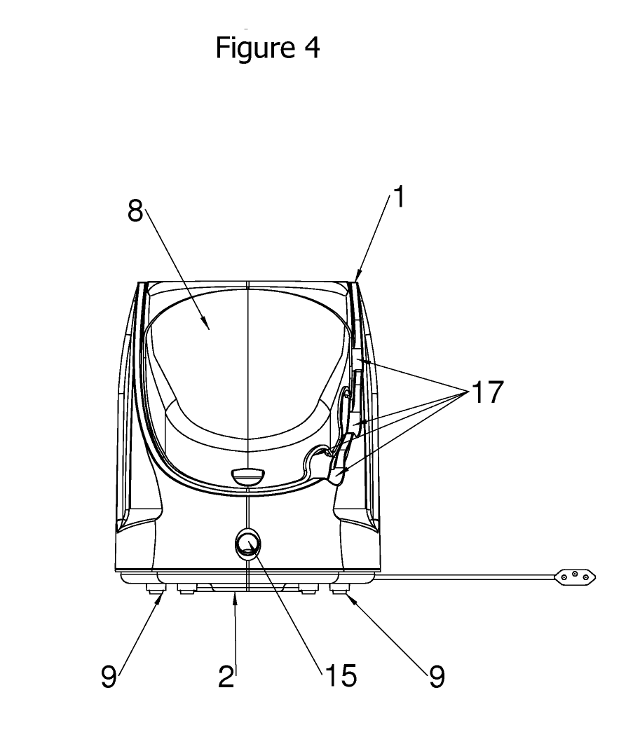

FIG. 5 illustrates the apparatus of the invention in the electronic version in right side view.

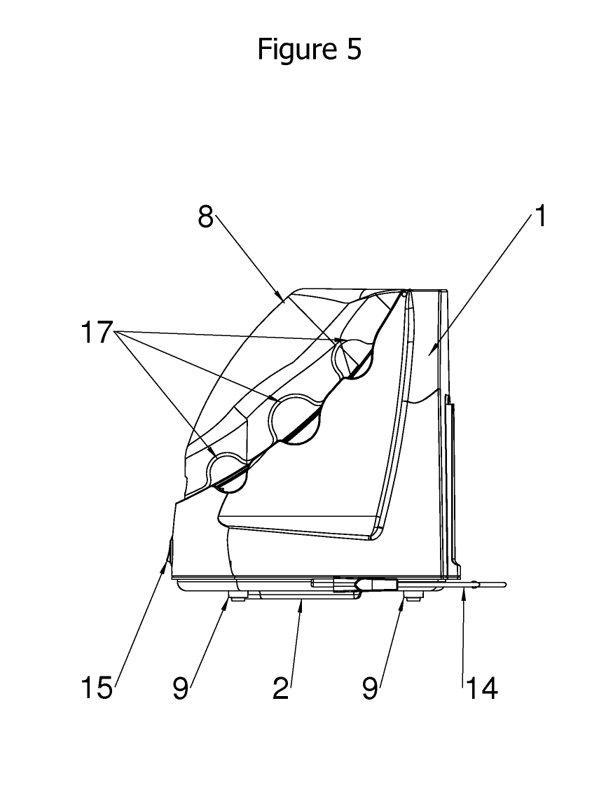

FIG. 6 illustrates the apparatus of the invention in the electronic version in left side view.

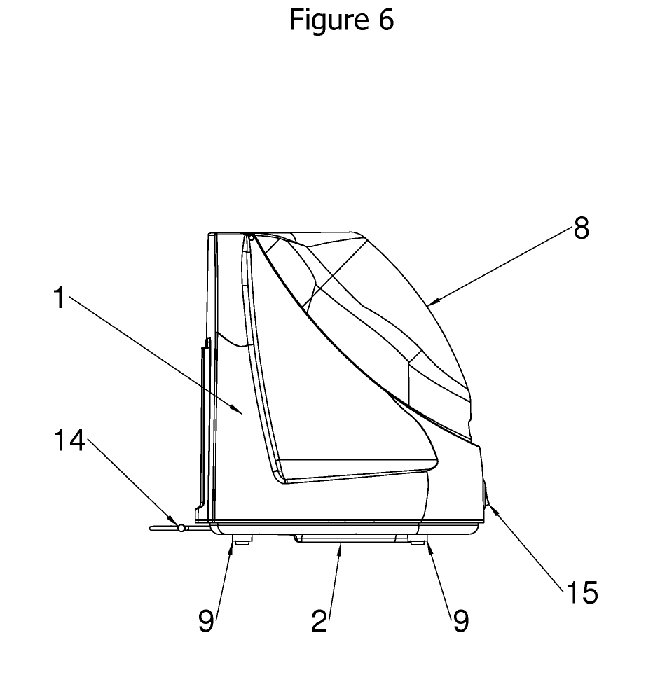

FIG. 7 illustrates the apparatus of the invention in the electronic version in rear view.

FIG. 8 illustrates the apparatus of the invention in the electronic version in upper view.

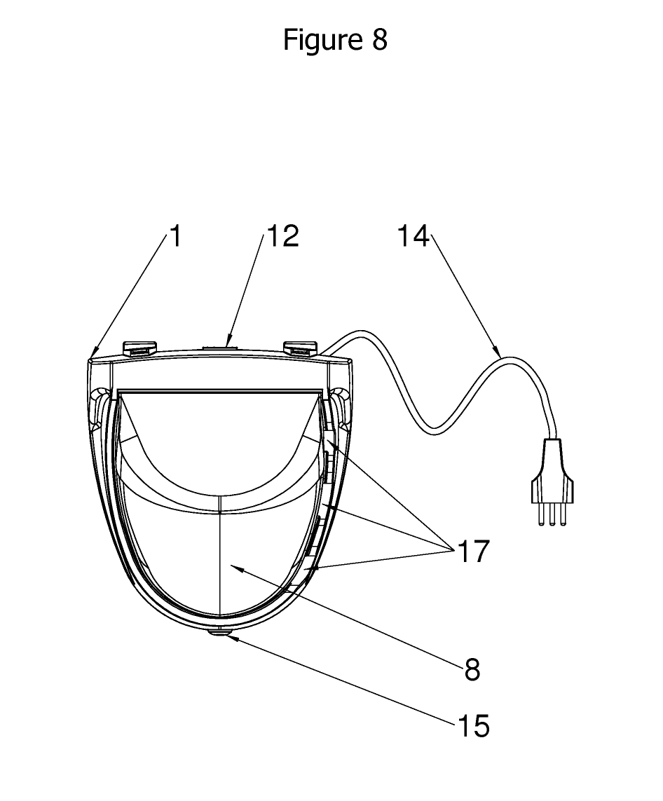

FIG. 9 illustrates the apparatus of the invention in the electronic version in lower view.

FIG. 10 illustrates the apparatus of the invention in the electronic version in right perspective view without the lid and with the brushes properly placed in the concave fits.

FIG. 11 illustrates the apparatus of the invention in the mechanic version in front view.

FIG. 12 illustrates the apparatus of the invention in the mechanic version in expanded right perspective view.

FIG. 13 illustrates the apparatus of the invention in the mechanic version in right perspective view.

### DETAILED DESCRIPTION OF THE PREFERRED EMBODIMENTS

According to the present invention, the term “brushes” means devices used for work in veterinary or human hair, such as brushes, combs, instruments and accessories that need to be cleaned periodically. The term thus includes any instruments such as scissors, etc., which require disinfecting and sanitizing.

The steam used for disinfecting and sanitizing is pure steam.

In order to ensure the disinfecting, products must be kept in contact with steam for four minutes.

As the apparatus takes about two minutes to reach the temperature required for steaming, the operation cycle total time is of six minutes.

Before the operation start, the operator must check if there is 100 ml of water in the tank.

Water is introduced by hand in the apparatus and refilled at each disinfecting cycle.

Brushes are inserted into the apparatus with the handles faced outside the apparatus in a horizontal position, preventing that the cables are heated and accidents occur to the user's hands.

To allow these advantages the apparatus of the invention comprises one steam chamber internally provided with a support with three concave housings, one housing for each brush or other similar instrument. The chamber, together with the case and lid, allows the said brushes of different sizes and shapes become arranged in a horizontal position in contact with the steam emanating from the chamber

The generated steam in the chamber comes into contact with the brushes to be cleaned and disinfected from bottom to top direction.

The case of the apparatus of the invention is molded in an engineering plastic, heat and impact-resistant. The case can have any color.

The case geometry of the present apparatus is not critical, but it must consider the arrangement need of the brushes and other products inside thereof such that all products receive the steam generated evenly.

An embodiment of the apparatus of the invention involves electronic controls.

Another embodiment of the apparatus involves mechanic controls.

The electronic embodiment features a control and operation electronic system and operation with electronic circuit board and sensor.

The mechanic embodiment is provided with a timer.

Both embodiments include a protection system comprising a thermostat.

The invention is described below related to the Figures attached, without, therefore, to be inferred that they are limitative of the invention. Rather, it should be clear for those skilled in the art that various modifications and variations are possible within the scope of the invention, this being enclosed only by the appended claims.

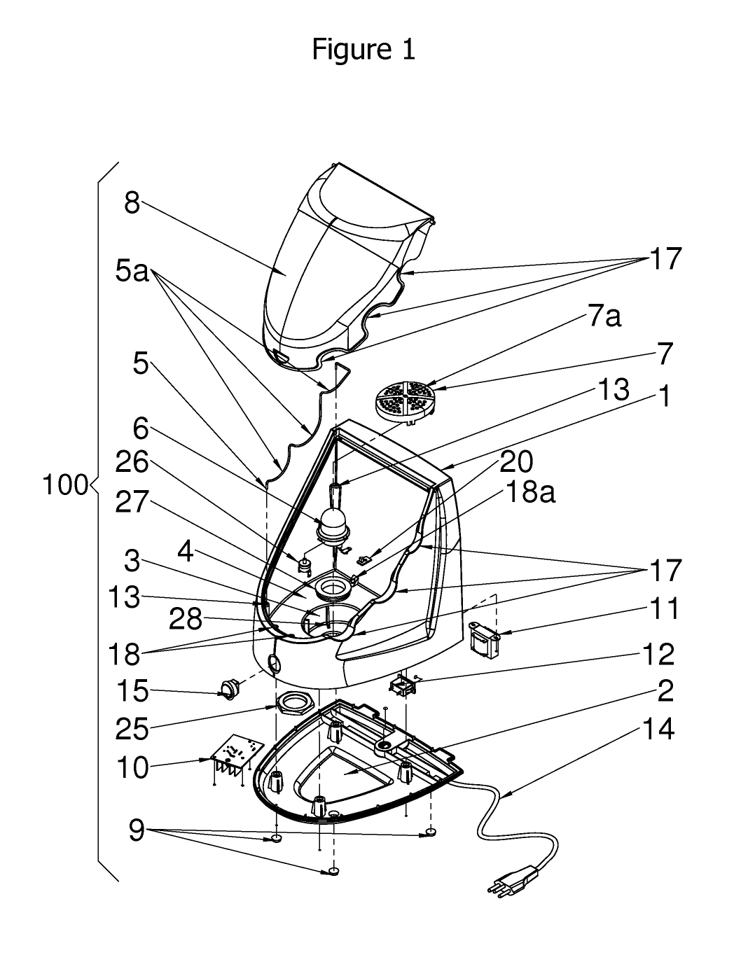

The apparatus in its electronic version, generally designed by the number (100) according to FIG. 1, consists of a case (1); base (2) of the case (1) on support feet (9); water tank (3); chamber (4); support (5) for brushes (16); electric resistance heating system (6); protection screen (7); tilting lid (8); thermostat (26); control and operation electronic system provided with the electronic circuit board (10); transformer (11); fuse holder (12); power cable (14); on/off switch (15); blue LEDs (18); red LEDs (18a) and sensor (20).

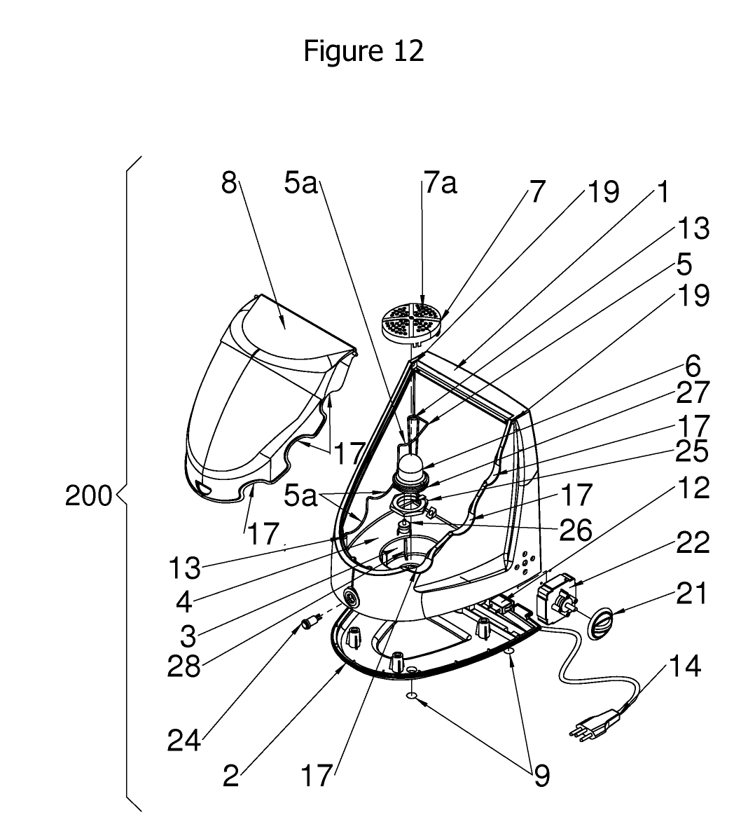

The apparatus in its mechanic version, generally designed by the number (200) (FIG. 12) consists of a case (1); base (2) of the case (1); water tank (3); chamber (4); support (5) for brushes (16); electric resistance heating system (6); protection screen (7); tilting lid (8); supports (9); thermostat (26); silicon ring (27); locknut (25) of the resistance (6); timer (22) button (21); fuse holder (12); power cable (14); and, pilot lamp (24).

The apparatus, either in the electronic (100) or in the mechanic (200) version, comprises a chamber (4) with steam system. The steam is produced through the shielded resistance contact (6) with the water present in the tank (3) of the present apparatus, as can be noted in FIGS. 1 and 12.

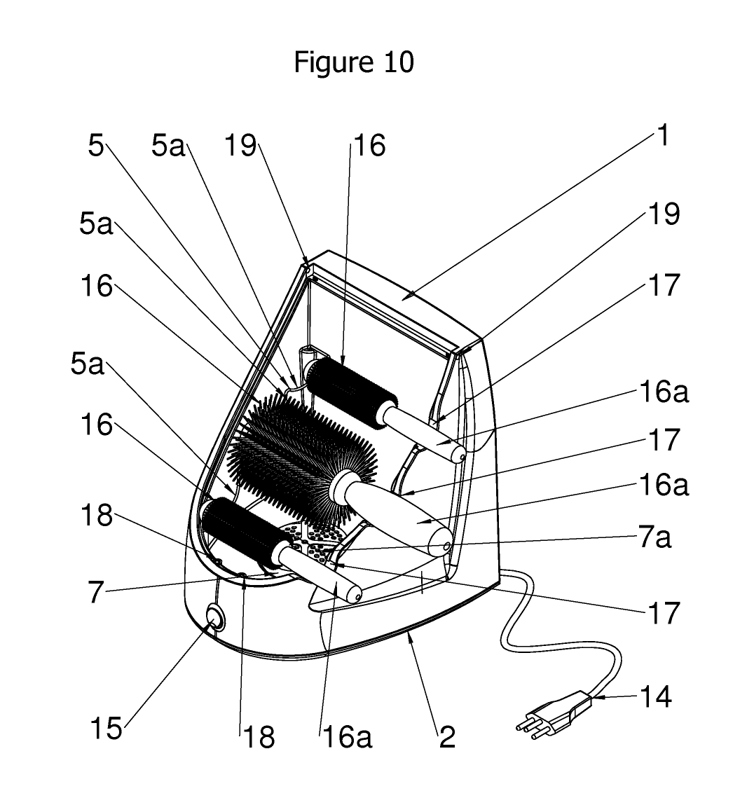

The apparatus (100, 200) allows inserting up to three brushes (16) for hair, combs, instruments and accessories, as illustrates the FIG. 10. The said brushes are involved for about four minutes by the steam generated by the apparatus (100, 200) at about 75° C. This procedure eliminates the microorganisms and bacteria sensitive to the sanitizing and disinfecting process.

The support (5) for brushes body supports up to three units at the same time for each disinfecting and sanitizing cycle.

The support (5) is designed in steel or any other material that does not deteriorate with moisture, has rod-molded format with concave fits (5a) for brushes (16) handles (16a). The support (5) is fitted inside the disinfecting and sanitizing chamber (4) in housing (13).

Another support is formed with the chamber (4) itself, case (1) and tilting lid (8) of the apparatus. The support thus formed is able to sustain the handles (16a) of the products (16) object of disinfecting through semicircular or concave accommodations (17) molded in the said component parts. Thus, handles (16a) protrude outside of the apparatus (100, 200) being free of heat, thereby avoiding the possibility of accidents for the operator and maintaining handles (16a) integrity.

Brushes arrangement (16) can be noted in FIG. 10.

The water tank (3) of the apparatus (100, 200) has capacity for about 100 ml of water intended to produce steam to disinfect and sanitize brushes and other products.

The tank geometry (3) is not critical, allowing circular and oval formats.

The tank base (3) is provided with fits (28) for screen insertion (7).

The screen (7) is provided with holes (7a) intended to allow a better temperature diffusion and any micro bubbles appearing on the resistance surface (6) during the tank (3) water heating.

The tilting lid (8) is transparent, in order to make visible the lighting of the LEDs (18a) intended to warn occurrence of overheating.

One way to produce steam by the apparatus (100, 200) is through an electrical resistance (6). The tank (3) has a through hole (not shown) to fit the electrical resistance (6), preferably shielded. The resistance (6) is properly stopped with a high temperature resistant silicon seal (27). The electric resistance (6) and the seal (27) are attached to each other with the aid of the nut (25).

Other stem production modes in the apparatus (100, 200) include a usual resistance or sealed (or encapsulated) resistance or by electrodes.

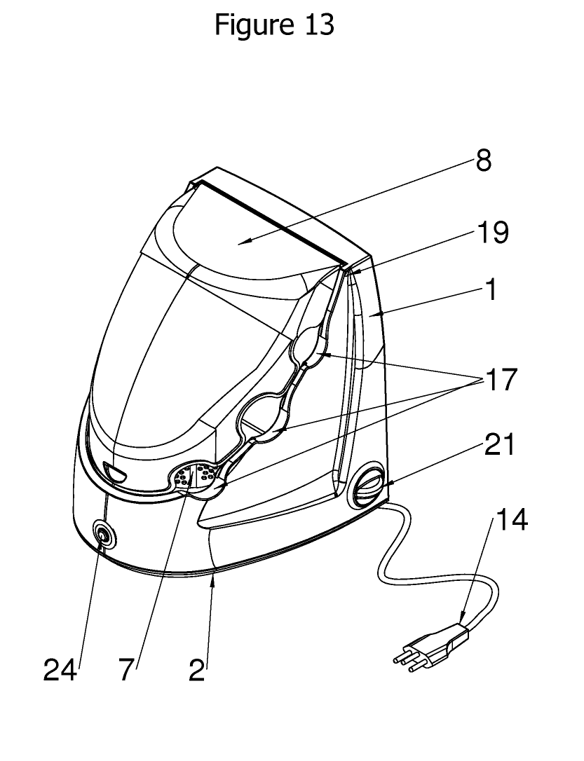

The case upper part (1) of the apparatus (100, 200) is provided with two holes (19), the said holes being responsible by the tilting lid (8) fit, as can be noted in FIGS. 10, 12 and 13.

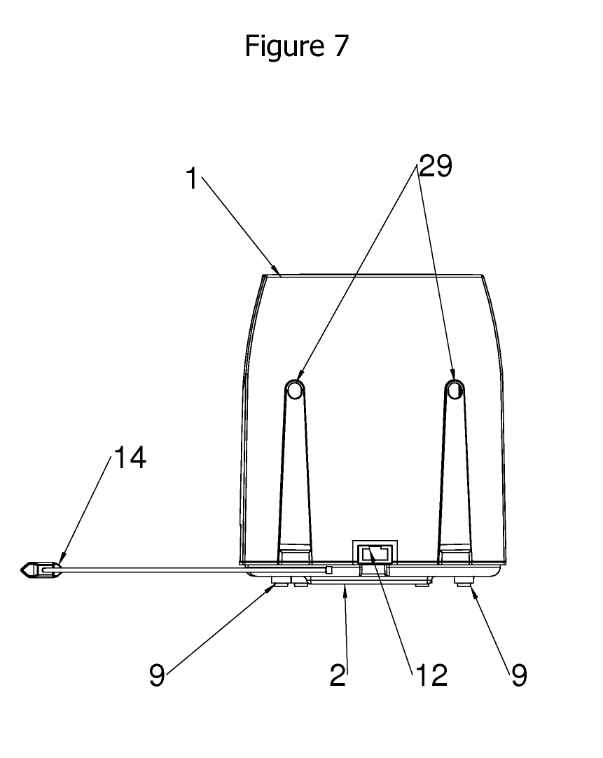

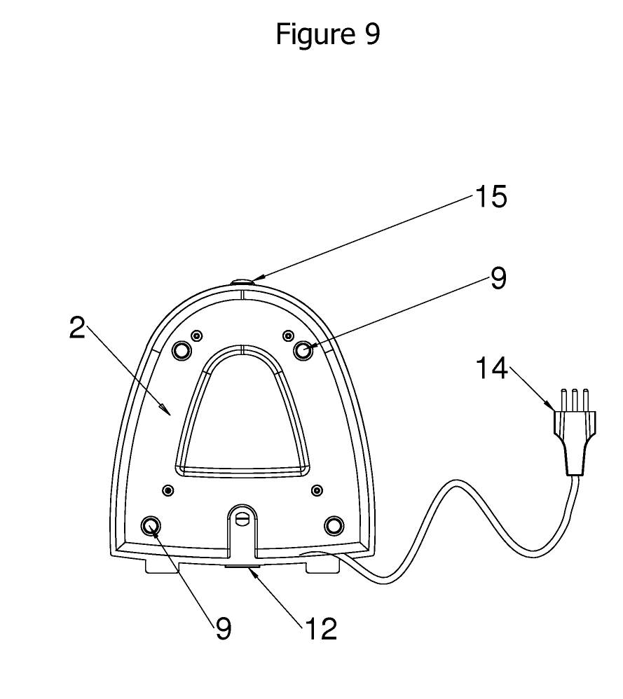

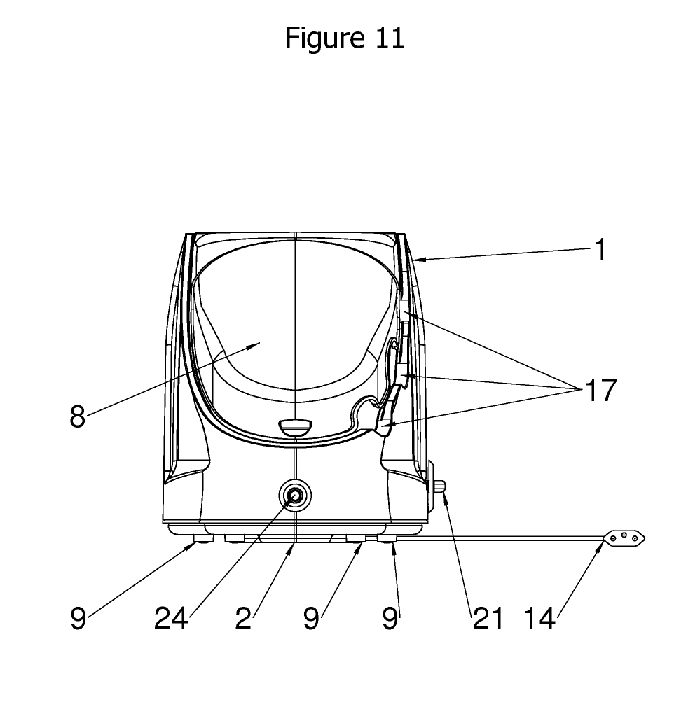

The base (2) of the apparatus (100, 200) is fitted and attached by screws in the case (1), so that the lower part of the said base is provided with rubber supports (9) without, however, being limited to this, as can be noted in FIGS. 7, 9 and 11.

In the electronic version (100), between the base (2) and the chamber (4) the control and operation electronic system is inserted. This system comprises electronic circuit board (10), transformer (11), fuse holder (12), power cable (14), thermostat (26), on/off switch (15), blue LEDs (18), red LEDs (18a) and sensor (20), as can be noted in FIG. 1.

In the mechanic version (200), between the base (2) and the chamber (4) the control system is inserted, comprising thermostat (26), timer (22), fuse holder (12), button (21) provided with pilot lamp (24), and power cable (14), as can be noted in FIG. 12.

In the rear part of the apparatus, either in the electronic (100) or in the mechanic (200) version, two holes (29) for screws fits are foreseen or hooks that provide attachment to wall, as can be noted in FIG. 7.

The on/off switch (15) in the electronic version (100) is placed in the front part of the apparatus (110, 200) of the invention, so it can have a light of any color related to the on/off switch, indicating the apparatus is powered.

In the mechanic version (200) the on/off switch functionality (15) is replaced by a button (21) connected to a pilot lamp (24), as can be seen in FIG. 12.

Also, in the mechanic version (200) the pilot lamp (24) replaces the LEDs (18, 18a) and the timer (22) replaces the electronic circuit board (10) of the electronic embodiment.

The pilot lamp (24) remains light up only while the timer (22) and the resistance (6) are switched on. The pilot lamp (24), the resistance (6) and the thermostat (26) are switched on in series.

Inside the chamber (4), in the electronic version (100), 2 blue LEDs (18) and 2 red LEDs (18a) are presented, as illustrates FIGS. 1 and 10. Through the apparatus electronic, blue LEDs (18) light up alternately indicating the disinfecting process start, and at the end of the process LEDs (18) remain lighted up.

When lacking water inside the tank (3), the apparatus, in the electronic version (100), through red LEDs (18a) alternately blinks indicating that it is necessary to add water.

Also, if the apparatus is overheated, red LEDs (18a) will light up.

Additionally in the electronic version (100) of the apparatus, in the case of: i) lack of water in the tank (3) or ii) overheating the security thermostat (26) stops the power supply to the shielded resistance (6).

The sensor (20) monitors the system temperature so the electronic circuit can manage the operation thereof. For example, if the sensor (20) notes an overheating, the electronic circuit will stop the apparatus (100) operation.

Analogous to the electronic version, also in the mechanic version (200) when missing water comes into operation the security thermostat (26), which interrupts the power supply for the shielded resistance (6)

In the case of overheating of the mechanic version (200) is equally the security thermostat (26) that stops the power supply for the shielded resistance (6).

The water supply inside the tank (3) is performed through any batcher container having 100 ml of filtered water that is discharged towards the protection screen (7). For this purpose, the lid (8) of the apparatus (100, 200), which must be empty, is opened and the water is added.

Once introduced to the apparatus (100, 200) brushes, combs or other products (16) to be disinfected and sanitized, the apparatus is switched on and the water is heated, generating steam.

The disinfecting and sanitizing of brushes, combs, instruments and accessories is started after two minutes, which is the time required for the chamber (4) of the apparatus (100, 200) reaches 75° C. After the temperature is reached, the apparatus (100, 200) takes approximately four minutes to complete the products disinfecting and sanitizing cycle.

Brushes, combs, instruments and accessories (16) are thus recovered from the apparatus (100, 200) securely for the operator and without any damage to the handles (16a) of said brushes (16) or similar products undergone to disinfecting and sanitizing.

1. Apparatus for disinfecting and sanitizing brushes, comprising a case (1) covered by a tilting lid (8) and supported by a base (2), and, inside the said case (1), a water tank (3), a chamber (4) with system for steaming the water contained in the tank (3), a support (5) for brushes (16), a shielded resistance heating system (6) for heating the water, a protection screen (7), a thermostat (26) and a control and operation system.

2. Apparatus according to claim 1, wherein the case upper part (1) is provided with two holes (19) intended for fitting the said tilting lid (8).

3. Apparatus according to claim 1, wherein the tilting lid (8) is transparent.

4. Apparatus according to claim 1, wherein the base (2) is fitted and attached by screws to the case (1), the lower part of the said base (2) being provided with supports (9).

5. Apparatus according to claim 1, comprising two holes (29) to fit the screws or hooks to attach in the wall.

6. Apparatus according to claim 1, wherein the chamber (4), together with the case (1) and the lid (8) allow the said brushes (16) to be arranged in the apparatus in a horizontal position, contacting the steam emanating from the bottom to the top from the said chamber (4).

7. Apparatus according to claim 1, wherein the steam needed to disinfect and sanitize the brushes (16) is produced thought the shielded resistance contact (6) with the water in the tank (3).

8. Apparatus according to claim 1, wherein each disinfecting and sanitizing cycle of the brushes (16) uses the total amount of water contained inside the tank (3).

9. Apparatus according to claim 1, wherein the support (5) for the brushes body (16) supports up to three units at the same time for each disinfecting and sanitizing cycle of the brushes (16).

10. Apparatus according to claim 1, wherein the support format (5) is a rod shaped with concave fits (5a) for handles (16a) of the brushes (16).

11. Apparatus according to claim 1, wherein the support (5) is fitted inside the chamber (4) in housing (13).

12. Apparatus according to claim 1, additionally comprising a support shaped with the proper chamber (4), case (1) and tilting lid (8) thereof, the said support being used to hold the handles (16a) of the brushes (16) through semicircular or concave accommodations (17) molded in the chamber (4), case (1) and lid (8) such that the handles (16a) protrude outside the apparatus and are free of heating.

13. Apparatus according to claim 1, wherein the water tank base (3) is provided with fits (28) for the screen insertion (7) provided with holes (7a) for better temperature diffusion during the water heating of the said tank (3).

14. Apparatus according to claim 1, wherein the shielded resistance (6) is dully stopped with high temperature resistant silicon seal (27).

15. Apparatus according to claim 1, wherein the control and operation system is electronic.

16. Apparatus according to claim 15, wherein the said system i) is inserted between the base (2) and the chamber (4) of the apparatus; and ii) comprises the electronic circuit board (10), transformer (11), fuse holder (12), power cable (14), thermostat (26), on/off switch (15), two blue LEDs (18), two red LEDs (18a) and sensor (20).

17. Apparatus according to claim 15, wherein the beginning of the disinfecting and sanitizing process is indicated by lighting up the blue LEDs (18) and the end of the process is indicated by keeping the LEDs (18) on.

18. Apparatus according to claim 15, wherein the lack of water or overheating inside the tank (3) is indicated by alternated blinking of the red LEDs (18a).

19. Apparatus according to claim 15, wherein in the case of i) lack of water in the tank (3) or ii) overheating the security thermostat (26) stops feeding power into the shielded resistance (6).

20. Apparatus according to claim 1, wherein the control and operation system is mechanic.

21. Apparatus according to claim 20, wherein the said system i) is inserted between the base (2) and the chamber (4) and ii) comprises the thermostat (26), button (21) connected to the pilot lamp (24), timer (22), fuse holder (12) and power cable (14).

22. Apparatus according to claim 20, wherein the pilot lamp (24) remains on only when the timer (22) and the shielded resistance (6) are on.

23. Apparatus according to claim 19, wherein the pilot lamp (24), the shielded resistance (6) and the thermostat (26) are connected in series.

24. Apparatus according to claim 20, wherein in the case of i) lack of water in the tank (3) or ii) overheating the security thermostat (26) stops feeding power to the shielded resistance (6).
